(19) United States
(12) Patent Application Publication
(10) Pub. No.: US 2011/0086010 A1
(43) Pub. Date: Apr. 14, 2011

(54) Methods, Systems, and Apparati for Cellular Therapeutic Agent Preparation and Delivery

(76) Inventors: Robert E. Newman, Sykesville, MD (US); Roman M. Salasznyk, N. Bethesda, MD (US)

(21) Appl. No.: 12/936,444

(22) PCT Filed: Apr. 23, 2008
(86) PCT No.: PCT/US08/61220

§ 371 Date: Dec. 16, 2010

(51) Int. Cl. A61K 35/32; A61K 35/12; A61P 29/00; A61P 17/02; A61M 5/31

(52) U.S. Cl. 424/93.7; 604/92

(57) ABSTRACT

Methods and devices for the preparation of a biological pharmaceutical composition outside of a classified environment, such as Biological Safety Cabinet type environment, are described. In one aspect of the present technology, a method is provided to prepare a biological pharmaceutical composition preferably in an area next to a patient's bedside or at a facility's pharmacy. The method comprises transferring an appropriate amount of a diluent from a diluent component (645) into a mixing receptacle (605) using a transfer device (620), connecting a pooling (630) harness between a biological pharmaceutical component (640) and the mixing receptacle, transferring a biological pharmaceutical composition through the pooling harness to the mixing receptacle, mixing the diluent and the biological pharmaceutical composition to form a biological pharmaceutical composition mixture and administering the biological pharmaceutical composition mixture to a patient for either flat based or weight based dosing needs.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND

The presently described technology generally relates to the preparation, and/or delivery of cellular therapeutic agents for patient (human or animal) administration. More specifically, the present technology relates to the preparation of and delivery systems apparatus for one or more therapeutic agents (e.g., mesenchymal stem cells) in an unclassified environment such as outside of a Biological Safety Cabinet (“BSC”).

Stem cells are a unique class of cells that can self-replicate and differentiate into multiple tissue types. These cells are generally classified according to their differentiation potential, or ability to become distinct cell types.

In the human adult, for example, two major classes of stem cells exist in the bone marrow, normally hematopoietic stem cells and mesenchymal stem cells. Throughout life, hematopoietic stem cells, or HSCs, located within the bone marrow give rise to most types of blood cells. HSC transplantation has served as the basis for a number of aggressive treatments for various types of cancer. However, therapies based on HSCs are largely limited to hematological disorders because HSCs can only differentiate into blood cells.

In contrast to HSCs, mesenchymal stem cells, or MSCs, are multipotent or pluripotent cells that can differentiate into various types of tissues, such as, for example, bone, muscle, fat, tendon, ligament, cartilage and bone marrow stroma when they receive appropriate biochemical and biomechanical signals. Additionally, other biochemical stimuli may cause MSCs to mobilize to areas of injury or inflammation. Once there, MSCs perform immunomodulatory activities, reduce inflammation, and coordinate tissue regeneration at a local level by producing tissue growth factors and by interacting with local cells to reduce inflammatory outcomes and scarring.

Conventional modes of administration for the delivery of mesenchymal stem cell compositions include systemic intravenous injection and injection directly to the intended site of activity. These particular MSC compositions can be administered by any convenient route, for example, by infusion or bolus injection and can be administered together with other biologically active agents.

Further, mesenchymal stem cells can be administered alone; however, preferably, the mesenchymal stem cells are utilized in the form of pharmaceutical compositions. Such compositions contain a therapeutically effective amount of the mesenchymal stem cells, and a pharmaceutically acceptable carrier or excipient. Such a carrier may be a medically relevant diluent such as an electrolyte solution, saline, buffered saline, dextrose, water, or combinations thereof.

Moreover, a mesenchymal stem cell composition as a pharmaceutical formulation usually is formulated to be prepared in a classified environment that satisfies the International Standards Organization (“ISO”) class 5 or equivalent standard, such preparation within a Biological Safety Cabinet. Generally, these types of MSC-based pharmaceutical formulations are supplied as a cryopreserved concentrate in a hermetically sealed container indicating the quantity of active agent. When the formulation is to be administered by infusion, for example, it can be dispensed via an infusion bag containing sterile pharmaceutical grade water, and a medically relevant diluent or saline. This particular composition preparation is usually performed in a classified environment to prevent contamination, such as, again, within in a Biological Safety Cabinet. Further, such preparation is usually performed in a stem cell laboratory apart from the typical preparation areas, for example, hospital pharmacies or near the patient's bedside or delivery area.

When the mode of administration is by infusion, the typical method of preparation for MSC-based pharmaceutical compositions, used heretofore, was complicated and required the preparation steps to be performed in a BSC. The conventional method of preparation for MSC-based pharmaceutical compositions for infusion included the steps of thawing the biological pharmaceutical composition, attaching a coupler, such as a Cobe coupler, to the diluent bag, to the infusion bag, and to the biological pharmaceutical bag. A syringe was used to transfer the diluent to the biological pharmaceutical bag for mixing. Attachment of the syringe to the bags was accomplished through the use of couplers. After the diluent and the biological pharmaceutical were mixed in the biological pharmaceutical bag, the mixture was transferred to an infusion bag. This complicated, multi-step process is repeated for subsequent units of the particular biologic pharmaceutical formulation desired. The infusion bag was then removed from the classified environment and transferred to an infusionist for administration to the patient. Again, this entire procedure, except for thawing, had to be performed in a classified environment such as a BSC to insure that the steps were performed in a sterile environment to prevent contamination.

Further, the conventional method(s) of preparation of MSC-based pharmaceutical compositions was time consuming, thus causing inefficient use of professional personnel and facilities, and resulted in increased cost.

Thus, a need exists for a biological pharmaceutical preparation (such as an MSC-based pharmaceutical composition) method that reduces or prevents the chance for contamination of the MSC-based pharmaceutical composition; can be performed outside of a classified, BSC-type environment; utilizes a simplified procedure; and reduces the preparation time needed for conventional methods.

There is also a need for a biological pharmaceutical preparation method that provides for dilution of a cellular biological product allowing for both flat based dosing where every patient is administered the same amount of cells regardless of weight as well as a method for weight based dosing where patients are administered a specific amount of cells based on their weight using the same preparation technique.

It would also be desirable to have a biological pharmaceutical preparation method that provides for the mixture of one or more drug delivery container/receptacles in a single preparation, thereby improving the efficiency of the preparation process.

### BRIEF SUMMARY OF THE PRESENT TECHNOLOGY

In general, at least one aspect of the presently described technology provides one or more methods for the preparation of a biological pharmaceutical composition, in particular, an MSC-based pharmaceutical composition (e.g., multipotent or pluripotent cells such as human MSCs or differentiated cells), outside of a BSC-type preparation environment.

Another aspect of the presently described technology in where one or more methods and/or systems to reduce the time required for preparing a biological pharmaceutical composition (e.g., multipotent or pluripotent cellular pharmaceutical compositions of the present technology) for administration to a human or an animal patient.

A further aspect of the presently described technology is to provide one or more methods and/or devices to simplify the procedure required to prepare a biological pharmaceutical preparation for administration to a human or an animal patient.

A still further aspect of the presently described technology comprises one or more methods for pharmaceutical composition preparation of a biological pharmaceutical composition (e.g., MSC-based pharmaceutical compositions of the present technology) for human or animal patient administration outside of a controlled BSC-type environment, yet possible contamination is prevented or avoided. In one embodiment of this aspect, the method comprises the steps of transferring an appropriate amount of a diluent from a diluent component into a mixing receptacle

thawing a frozen biological pharmaceutical composition contained in a receptacle component, draining the diluent into the receptacle containing the biological pharmaceutical component, transferring the biological pharmaceutical composition and the diluent to the mixing receptacle to form a biological pharmaceutical composition mixture, and administering an appropriate amount of the biological pharmaceutical composition mixture to a human or an animal patient.

Another aspect of the present technology comprises one or more kits for preparing biological pharmaceutical compositions (e.g., MSC-based pharmaceutical compositions of the present technology) for administration to a human or animal patient outside of a controlled BSC-type environment. At least one embodiment of this preparation kit aspect comprises a biological pharmaceutical composition receptacle component; a mixing receptacle component; a diluent component filled with an appropriate amount of a diluent; a diluent transfer device; a biological pharmaceutical composition conveying system and a human or animal administration component.

Another aspect of the present technology comprises a method for preparing a biological pharmaceutical composition (e.g., MSC-based pharmaceutical compositions of the present technology) wherein the biological pharmaceutical composition is mixed with a diluent outside of a classified environment to form a biological pharmaceutical composition mixture.

The aforesaid and other aspects and advantages of the present technology will become more apparent upon consideration of the one or more for the embodiments which are illustrated in the accompanying drawings, description and appended claims.

### BRIEF DESCRIPTION OF SEVERAL VIEWS OF THE DRAWINGS

FIG. 1 is flow diagram of at least one embodiment of the biological pharmaceutical composition preparation procedure of the present technology.

FIG. 2 is an illustration of at least one embodiment of the biological composition preparation procedure to transfer a biological pharmaceutical composition into a receptacle component outside of a controlled environment.

FIG. 3 is a flow diagram of at least one embodiment of biological pharmaceutical preparation of the present technology.

FIG. 4 is a flow diagram of at least one embodiment of the biological pharmaceutical composition preparation of the present technology.

FIG. 5 is an illustration of at least one embodiment of the dilution method of present technology.

FIG. 6 is an illustration of at least one embodiment of the dilution method of present technology.

The foregoing summary, as well as the following detailed description of certain embodiments of the present technology, will be better understood when read in conjunction with the appended drawings, description and appended claims. For the purpose of illustrating the present technology, certain embodiments are shown in the drawings. It should be understood that the presently described technology is not limited in any manner or position to the arrangements and instrumentality shown in the attached drawings.

### DETAILED DESCRIPTION OF THE PRESENT TECHNOLOGY

Conventionally, preparations of biological pharmaceutical compositions for cellular therapies require that the biological pharmaceutical composition preparation be carried out in a controlled environment that satisfies the International Standards Organization (“ISO”) class 5 or equivalent, such as preparation within a Biological Safety Cabinet (“BSC”). In a preferred embodiment of the present technology described and claimed herein, a biological pharmaceutical composition preparation may be performed outside of the BSC-type environment.

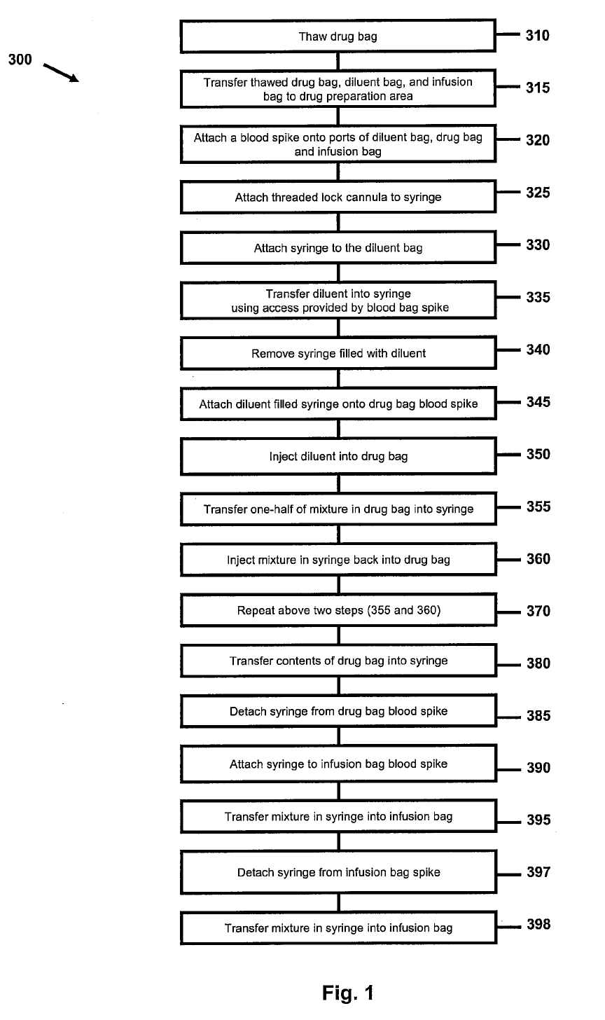

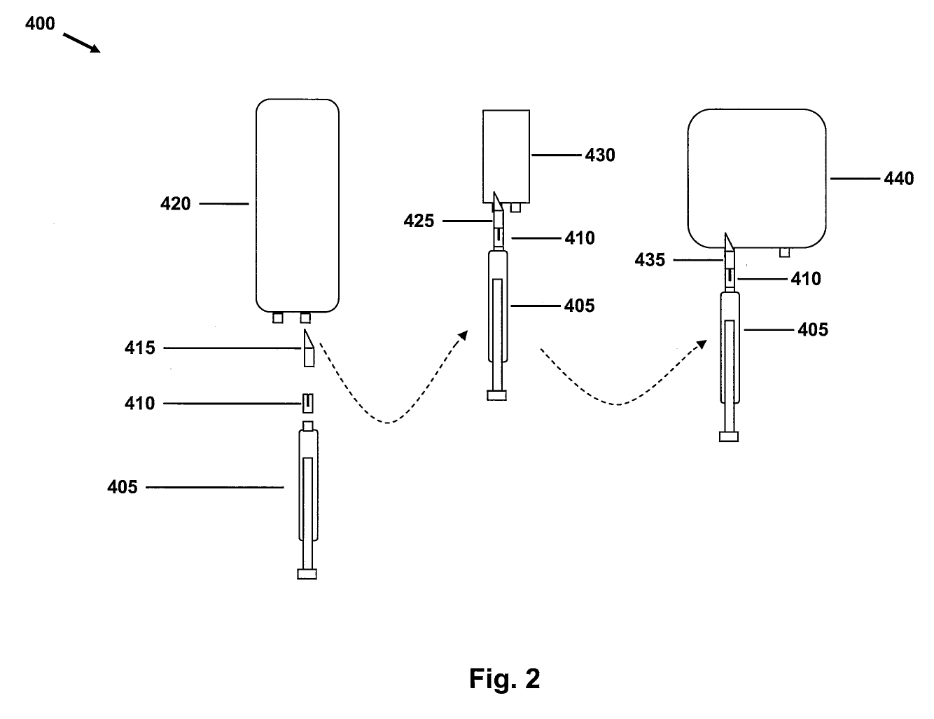

FIGS. 1 and 2 illustrate a flow diagram and a schematic of at least one embodiment of a method for the preparation of biological pharmaceutical compositions of the present technology, preferably MSC-based pharmaceutical compositions (e.g., multipotent or pluripotent cells such as human MSCs or differentiated cells), for infusion into human or animal patients as cellular therapies. Methods 300, 400 illustrate the preparation of the present technology to be carried out outside of a controlled environment, for example, outside of a BSC. The method of pharmaceutical preparation may be performed in an area next to a human or an animal patient. A biological pharmaceutical component 430 containing a biological pharmaceutical composition may be received at an administering facility or biological pharmaceutical preparation facility as a cryopreserved concentrate. Preferably, the biological pharmaceutical preparation in this embodiment can be performed at a facility's pharmacy or at an area near the patient (e.g., by the patient's room or bedside). At step 310, a cryopreserved biological pharmaceutical component 430 containing a biological pharmaceutical is thawed, preferably in a 37°±15° C. water bath. It is contemplated that other mediums may be used to thaw the frozen biological pharmaceutical compositions, such as air, liquids, semi-liquids, such as gels, or solids, such as hot plates.

At step 315, the thawed biological pharmaceutical component 430, a diluent component 420, and a mixing receptacle 440 may be transferred to an area for mixing, such as an area near the patient or to a facility's pharmacy (see FIGS. 1 and 2). It is contemplated that other preparation areas may be used to prepare the biological pharmaceutical composition outside of a BSC, such as, in the case of a human patient, the patient's bedside. As an example, the appropriate biological pharmaceutical composition in the thawed biological pharmaceutical component 430 may be MSCs, preferably human MSCs. It will be appreciated by those skilled in the art that the present technology envisions the use of multipotent or pluripotent cells and differentiated cells. For example, MSCs are multipotent or pluripotent cells that can differentiate into various types of tissues/cells such as bone, muscle, heart, fat, tendon, ligament, cartilage, bone marrow stroma, among others when exposed to an appropriate biochemical and/or chemical stimuli, signal, trigger, or combinations thereof. Additionally, other biochemical stimuli may cause MSCs to mobilize to areas of injury or inflammation areas as that caused by an inflammatory disease/disorder.

The diluents in the diluent component 420 may be a medically relevant diluent such as an electrolyte solution, saline, buffered saline, dextrose, water, or combinations thereof. Examples of the mixing receptacle 440 may be syringes or an infusion bag attached to a pooling harness.

At the biological pharmaceutical preparation area, for example, at step 320, a plurality of connector elements 415, 425, 435, may be attached to the biological pharmaceutical component 430, the diluent component 420 and the mixing receptacle 440, respectively. In FIGS. 1 and 2, the connector elements 415, 425, 435 are depicted as a blood spike. It is contemplated that any device that prevents or avoids contamination and can be repeatedly used without compromising the sterility of the contents of the components or receptacles may be substituted as the connector elements. An example of connectors may be an Interlink blood bag spike or a Cobe coupler. In step 325, a locking device 410 may be attached to a transfer device 405. In FIGS. 1 and 2, the locking device 410 is depicted as a threaded lock cannula. It should be understood that any locking device, such as a Cobe coupler, may be used that can be attached to the transfer device 405, and locks the transfer device 405 to the biological pharmaceutical component 430, the diluent component 420 or the mixing receptacle 440. At steps 330 and 335, the transfer device 405 is preferably attached to the diluent component 420 and an appropriate amount of the diluent may be transferred into the transfer device 405. The appropriate amount of diluent may range from 0 ml to about 1000 ml.

In FIGS. 1 and 2, the transfer device 405 is depicted as a syringe. It is contemplated that the transfer device 405 may be any device, such as, for example, a flexible tubular transfer element that may be used to transfer the diluent from the diluent component 420 outside of a BSC-type environment. Other examples of transfer devices 405 may be a flexible tubing element such as a pooling harness or a rigid device. At steps 340 to 350, it is contemplated that the transfer device 405 may be disconnected from the diluent component 420 and connected to the biological pharmaceutical component 430 and the diluent transferred into the biological pharmaceutical component 430.

Next, at steps 355 through 370, preferably a portion of the biological pharmaceutical composition mixture in biological pharmaceutical component 430 is transferred back into the transfer device 405 and then transferred back into the biological pharmaceutical component 430. The range of the portion of the biological pharmaceutical composition mixture that may be transferred may range from no transfer of any portion of the biological pharmaceutical composition mixture, to the transfer of all of the portion of the biological pharmaceutical composition mixture. It is contemplated that these steps may be omitted or performed a plurality of times depending on the desired infusion protocol utilized, based on the pharmaceutical component to be mixed and infused based upon current mixing and delivery guidelines.

At steps 380 through 395, it is contemplated that the biological pharmaceutical composition mixture contents of the biological pharmaceutical component 430 are transferred back into the transfer device 405. The transfer device 405 may then be detached from the biological pharmaceutical component 430. The transfer device 405 may then be attached to the mixing receptacle 440 and the biological pharmaceutical composition mixture contents of the transfer device 405 may be transferred into the mixing receptacle 440. At step 397, the transfer device 405 is preferably detached from the mixing receptacle 440. At step 398, the mixing receptacle 440 containing the biological pharmaceutical composition mixture may be transferred to the biological pharmaceutical administration area. It should be understood that the steps described above may be performed in an order other than that described above. Further, steps may be combined or performed at the same time. It should be understood, that if no diluent is used, steps 330 to 350 may be eliminated. Thus, a variety of procedures are envisioned and should be appreciated by those skilled in the art.

An appropriate amount of the biological pharmaceutical composition mixture will vary from patient to patient depending on various factors, such as, for example, the patient's weight. Also, the appropriate amount may be based on flat dosing where every patient is administered an amount of cells regardless of weight. As an example of flat dosing, 1, 2, 3 or 4 biological pharmaceutical components may be used for one infusion and the range of diluents may be from about 15 ml to about 1000 ml per biological pharmaceutical component used.

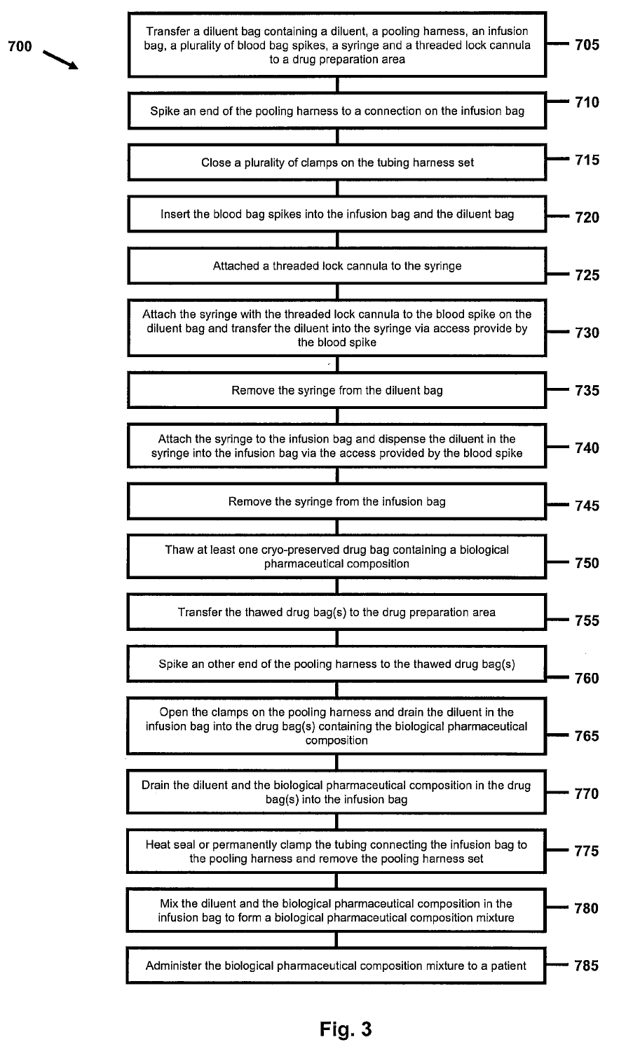

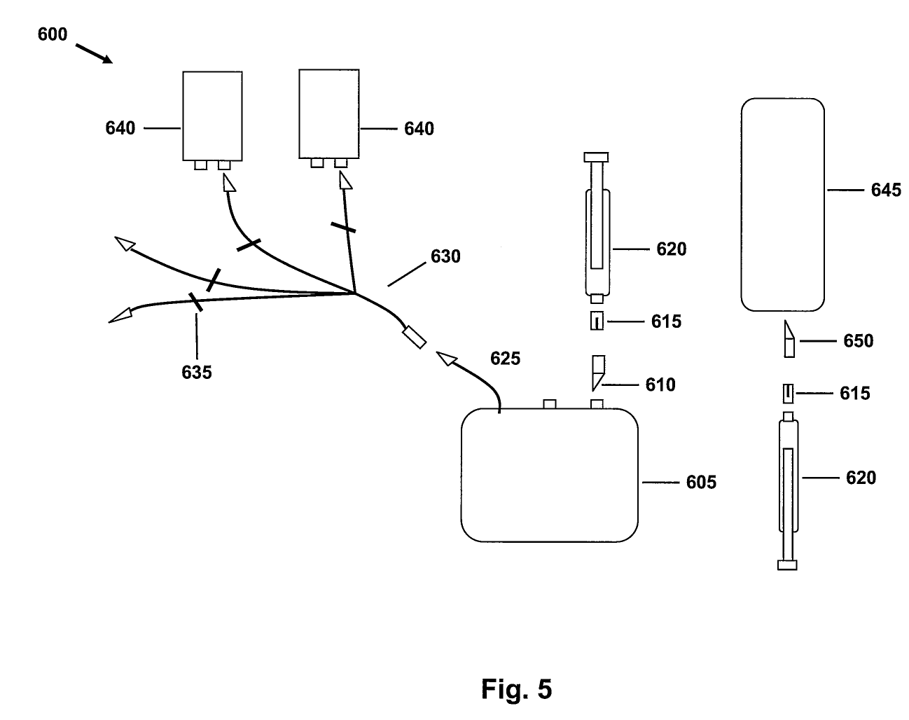

FIGS. 3 and 5 illustrate a flow diagram and a schematic of at least one embodiment of a method for the preparation of biological pharmaceutical compositions, preferably MSC-based pharmaceutical compositions, for infusion into human or animal patients as cellular therapies of the presently described technology. Methods 700, 600 illustrate the preparation of the present technology to be performed outside of a controlled environment, for example, outside of a BSC. The method of pharmaceutical preparation may be performed in an area next to a human or an animal patient. A biological pharmaceutical component 640 may be received at an administering facility or a biological pharmaceutical preparation facility as a cryopreserved concentrate. In this embodiment, it is contemplated that one or more than one biological pharmaceutical components 640 containing a biological pharmaceutical composition may be prepared for administration simultaneously using the method and apparatus disclosed.

At step 705, a diluent component 645, a mixing receptacle 605, a conveying system 630, a plurality of blood spikes 605, 615, a locking device 615, and a transfer device 620 may be transferred to a mixing area, such as an area near the patient. Diluents in the diluent component 645 may be a medically relevant diluent such as an electrolyte solution, saline, buffered saline, dextrose, water, or combinations thereof. The amounts of diluent may range from 0 ml to about 1000 ml.

At step 710, an end of the conveying system 630 is connected to the mixing receptacle 605 via a mixing receptacle connector element 625.

In FIG. 5, the conveying system 630 is depicted as a multi-lead multiple tubing system, such as, for example, a pooling harness set for pooling a plurality of the biological pharmaceutical compositions through tubing that is compatible with sterile connection technology. However, alternatives are envisioned for use in practice of the presently described technology. For example, a 4 to 1 spike adaptor or a 5 to 1 spike adaptor are contemplated among others. These may be flexible tubing or rigid tubing elements. The conveying system 630 may consist of one transfer conveyor or more than one transfer conveyor to allow for a plurality of biological pharmaceutical components 640 containing the biological pharmaceutical compositions to be connected for transfer to the mixing receptacle 605 and allow for the sterile connection and transfer of the biological pharmaceuticals compositions to the mixing receptacle 605.

At step 715, the conveying system 630 is closed using at least a clamp 635 on the transfer harness 630. At steps 720 through 730, it is contemplated that a second connector element 610 may be connected to the mixing receptacle 605 and a first connector element 650 may be connected to the diluent component 645. Further, the locking device 615 may be connected to the transfer device 620. The transfer device 620 with the locking device 615 may be attached to the first connector element 650 that is attached to the diluent component 645. In FIG. 5, the first connector element 650 and the second connector element 610 are depicted to be a blood spike. It is contemplated that any device that prevents or avoids contamination and can be repeatedly used without compromising the sterility of the contents of the diluent component 645 or the mixing receptacle 605 may be used as the first connector element 650 and a second connector element 610, such as an Interlink blood bag spike or a Cobe coupler. Also, in FIG. 5, the locking device 615 is depicted as a threaded lock cannula. It should be understood that any locking device, such as an Interlink threaded lock cannula, that can be attached to the transfer device 620 and locks the transfer device 620 to the diluent component 645 through the connector element 650 may be used.

After connecting the transfer device 620 to the diluent component 645, an appropriate amount of the diluent, which may be in the range of 0 ml to about 1000 ml, in the diluent component 645, may be withdrawn from the diluent component 645 into the transfer device 620. The appropriate amount of the diluent may vary for each patient. In the illustrated embodiment of FIG. 5, the transfer device 620 is depicted as a syringe. It is contemplated that any transfer device that allows for the sterile transfer of the diluent may be used, for example, a syringe, a flexible tube element, a pooling harness and a rigid tubing element.

In steps 735 through 745, the transfer device 620 may then be detached from the diluent component 645 and connected to the mixing receptacle 605 through the locking device 615 and the second connector element 610. The second connector element 610 is depicted as a blood spike in FIG. 5. It is contemplated that any device that prevents or avoids contamination and can be repeatedly used without compromising the sterility of the contents of the mixing receptacle 605 may be used as the second connector element 610, such as an Interlink blood bag spike or a Cobe coupler. The diluent in the transfer device 620 may be transferred to the mixing receptacle 605. The transfer device 620, may be removed from mixing receptacle 605. It is contemplated that any device that prevents contamination and can be repeatedly used without compromising the sterility of the contents of the mixing receptacle 605 may be used as the second connector element, such as an Interlink blood bag spike or a Cobe coupler.

At step 750, the cryopreserved biological pharmaceutical component 640 containing a biological pharmaceutical composition may be thawed preferably in a in a 37°±15° C. water bath. It is contemplated that other mediums may be used to thaw the frozen biological pharmaceutical compositions, such as air, liquids, semi-liquids, such as gels, or solids, such as hot plates. At step 755, the thawed biological pharmaceutical components 640 containing the biological pharmaceutical composition may be transferred to the drug preparation area. In step 760, an other end of the conveying system 630 is connected to the thawed biological pharmaceutical component 640, with the other end of the conveying system 630 previously connected to the mixing receptacle 605 via a mixing receptacle connector element 625 described in step 710. As an example, the appropriate biological pharmaceutical composition in the thawed biological pharmaceutical component 640 may be MSCs.

At step 765, after the connection of the conveying system 630 to the biological pharmaceutical components 640 and the mixing receptacle 605, the clamps 635 on the conveying system 630 may be opened and the diluent in the mixing receptacle 605 transferred to the biological pharmaceutical components 640. At step 770, the biological pharmaceutical compositions and the diluent in the biological pharmaceutical components 640 may be transferred back to the mixing receptacle 605. At step 775, after the contents in the biological pharmaceutical components 640 are transferred to the mixing receptacle 605, the mixing receptacle connector element 625 on the mixing receptacle 605 may be clamped closed or heat sealed and the transfer conveyor 630 may be removed.

In step 780, the diluent and the biological pharmaceutical compositions contents of the mixing receptacle 605 may then be mixed. In step 785, after mixing the diluent and the biological pharmaceutical compositions in the mixing receptacle 605 to form a biological pharmaceutical composition mixture, an appropriate amount of the biological pharmaceutical composition mixture may be administered to the patient. Examples of administration devices or techniques may be, at least, via an infusion pump, a syringe, or by gravity flow.

In this embodiment, an appropriate amount of the biological pharmaceutical composition mixture may vary from patient to patient depending of various factors, such as, for example, the patient's weight. To remove the biological pharmaceutical composition mixture for weight based dosing to achieve the correct volume immediately prior to the patient administration, the operator may use the transfer device 620 to withdraw the appropriate volume from the mixing receptacle 605 via the connector element 610. Also, the appropriate amount may be based on flat dosing where the patient is administered the same amount of cells regardless of weight.

Further, it should be understood that the steps described above may be performed in an order other than that described above. Also, steps may be combined or performed at the same time.

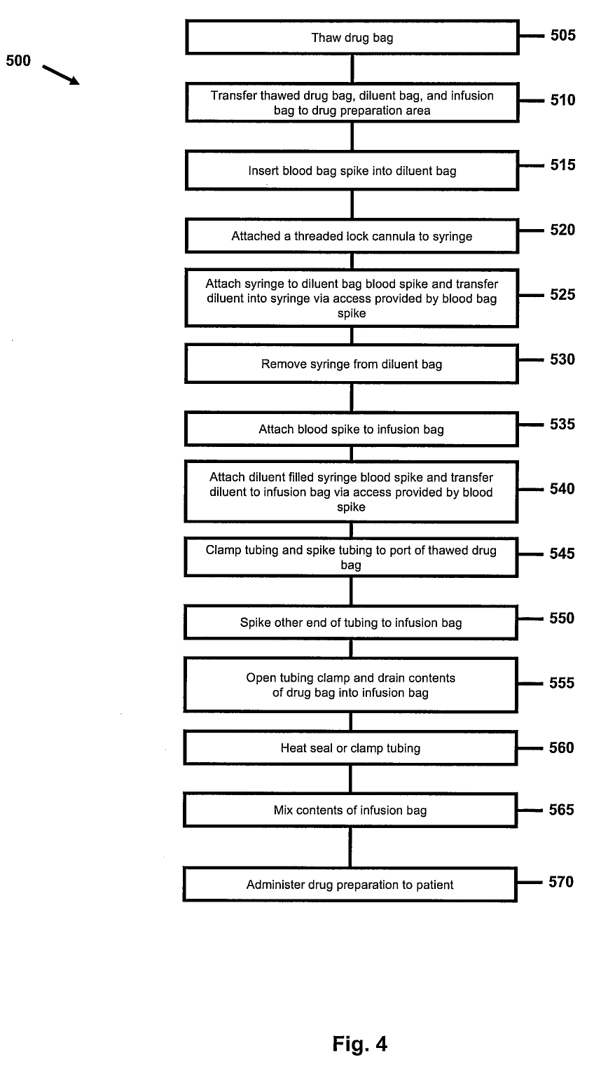

FIGS. 4 and 5 illustrate a flow diagram and a schematic of at least one alternative embodiment of a method for the preparation of biological pharmaceutical compositions, preferably MSC-based pharmaceutical compositions, for infusion into human or animal patients as cellular therapies of the presently described technology. Methods 500, 600 illustrate the preparation of the present technology to be performed outside of a controlled environment, for example, outside of a BSC. The method of pharmaceutical preparation may be performed in an area next to a human or an animal patient. A biological pharmaceutical component 640 may be received at an administering facility or a biological pharmaceutical preparation facility as a cryopreserved concentrate. In this embodiment, it is contemplated that one or more than one biological pharmaceutical components 640 containing a biological pharmaceutical composition may be prepared for administration simultaneously using the method and apparatus disclosed.

At step 505, the cryopreserved biological pharmaceutical component 640 containing a biological pharmaceutical composition may be thawed preferably in a in a 37°±15° C. water bath. It is contemplated that other mediums may be used to thaw the frozen biological pharmaceutical compositions, such as air, liquids, semi-liquids, such as gels, or solids, such as hot plates. At step 510, the thawed biological pharmaceutical components 640, a diluent component 645, and a mixing receptacle 605 may be transferred to a mixing area, such as an area near the patient. Diluents in the diluent component 645 may be a medically relevant diluent such as an electrolyte solution, saline, buffered saline, dextrose, water, or combinations thereof. The amounts of diluent may range from 0 ml to approximately 1000 ml. At steps 515 through 540, it is contemplated that a first connector element 650 is attached to the diluent component 645. Further, a locking device 615 is connected to the transfer device 620. The locking device 615 may then be attached to the first connector element 650 that is attached to the diluent component 645. In FIGS. 4 and 5, the first connector element 650 is depicted to be a blood spike. It is contemplated that any device that prevents or avoids contamination and can be repeatedly used without compromising the sterility of the contents of the diluent component 645 may be used as the first connector element, such as an Interlink blood bag spike or a Cobe coupler. Also, in FIGS. 4 and 5, the locking device 615 is depicted a threaded lock cannula. It should be understood that any locking device, such as an Interlink threaded lock cannula, that can be attached to the transfer device 620 and locks the transfer device 620 to the diluent component 645 through the connector element 650 may be used.

After connecting the transfer device 620 to the diluent component 645, an appropriate amount of the diluent, which may be in the range of 0 ml to approximately 1000 ml, in the diluent component 645, may then be withdrawn from the diluent component 645 into the transfer device 620. The appropriate amount of the diluent may vary for each patient. In the illustrated embodiments of FIGS. 4 and 5, the transfer device 620 is depicted as a syringe. It is contemplated that any transfer device that allows for the sterile transfer of the diluent may be used, for example, a syringe, a flexible tube element, a pooling harness and a rigid tubing element. The transfer device 620 may be detached from the diluent component 645 and connected to the mixing receptacle 605 through the locking device 615 and a second connector element 610. As above, the second connector element 610 is depicted as a blood spike. It is contemplated that any device that prevents or avoids contamination and can be repeatedly used without compromising the sterility of the contents of the mixing receptacle 605 may be used as the second connector element, such as an Interlink blood bag spike or a Cobe coupler. The diluent in the transfer device 620 may be transferred to the mixing receptacle 605. In steps 545 to 560, a conveying system 630 is closed using at least a clamp 635. One end of the conveying system 630 is connected to the biological pharmaceutical component 640 and the other end of the conveying system 630 is connected to the mixing receptacle 605 via a mixing receptacle connector element 625. As an example, the appropriate biological pharmaceutical composition in the thawed biological pharmaceutical component 640 may be MSCs. The diluent in the transfer device 620 may be transferred to the mixing receptacle 605. Any conveying system may be used that allows for the sterile transfer of the biological pharmaceutical from the biological pharmaceutical components 640 to the mixing receptacle 605.

In FIGS. 4 and 5, the conveying system 630 is depicted as a multi-lead multiple tubing system with clamps 635, such as, for example, a pooling harness set for pooling a plurality of the biological pharmaceutical compositions through tubing that is compatible with sterile connection technology. However, alternatives are envisioned for use in practice of the presently described technology. For example, a 4 to 1 spike adaptor or a 5 to 1 spike adaptor are contemplated. These may be flexible tubing or rigid tubing elements. The conveying system 630 may consist of one transfer conveyor or more than one transfer conveyor to allow for a plurality of biological pharmaceutical compositions to be connected for transfer to the mixing receptacle 605 and allow for the sterile connection and transfer of the biological pharmaceuticals to the mixing receptacle 605.

After connection of the conveying system 630 to the biological pharmaceutical components 640 and the mixing receptacle 605, the clamps 635 on the conveying system 630 may be opened and the biological pharmaceutical compositions in the biological pharmaceutical components 640 can be transferred to the mixing receptacle 605. In step 565, the contents of the mixing receptacle 605 may then be mixed. In step 565, after the contents in the biological pharmaceutical components 640 are transferred to the mixing receptacle 605, the mixing receptacle connector element 625 on the mixing receptacle 605 may then be clamped closed or heat sealed and the transfer harness 630 removed. In step 570, after mixing the diluent and the biological pharmaceutical compositions in the mixing receptacle 605 to form a biological pharmaceutical composition mixture, an appropriate amount of the biological pharmaceutical composition mixture may be administered to the patient. Examples of administration devices and/or procedures may be, at least, an infusion pump, a syringe, or via gravity flow.

An appropriate amount of the biological pharmaceutical composition mixture may vary from patient to patient depending of various factors, such as, for example, the patient's weight. To remove the biological pharmaceutical composition mixture for weight based dosing to achieve the correct volume immediately prior to the patient administration, the operator would use the transfer device 620 to withdraw the appropriate volume from the mixing receptacle 605 via connector element 610. Also, the appropriate amount may be based on flat dosing where every patient is administered the same amount of cells regardless of weight.

Further, it should be understood that the steps described above may be performed in an order other than that described above. Also, steps may be combined or performed at the same time.

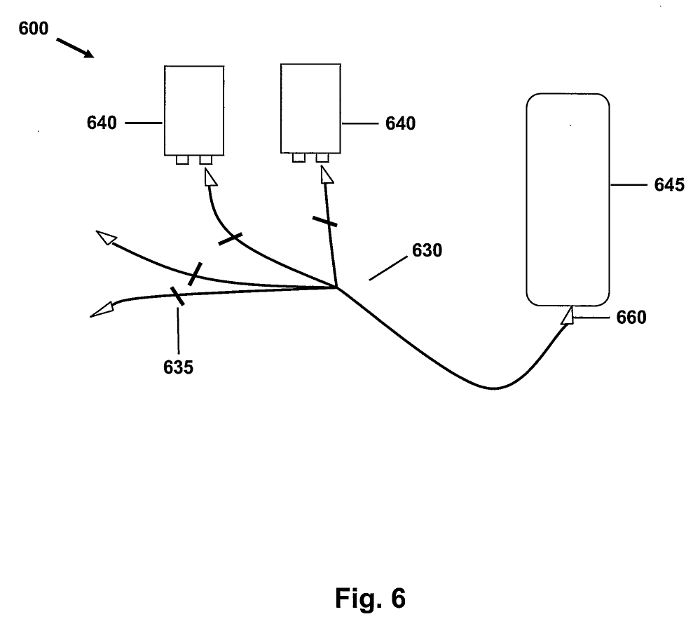

In an alternative embodiment, as shown in FIG. 6, it is contemplated that the diluent component 645 may be used in place of the mixing receptacle 605. In this embodiment, the contents in the biological pharmaceutical components 640 are transferred to the diluent component 645 through the conveying system 630. The conveying system 630 may be connected to diluent component 645 through connection part 660. It will be appreciated by those skilled in the art that connection part 660 may be any connector that allows sterile connection such as a blood spike or a coupler. After mixing the diluent and the biological pharmaceutical compositions in the diluent component 645 to form a biological pharmaceutical composition mixture, an appropriate amount of the biological pharmaceutical composition mixture now in the diluent component 645 may be administered to the patient. Examples of administration devices and/or procedures may be at least, an infusion pump, a syringe, or via gravity flow.

Another aspect of the present technology comprises one or more kits for preparing biological pharmaceutical compositions for administration to a human or animal patient outside of a controlled BSC-type environment. FIG. 5 illustrates one aspect of the preparation kit. The components of the kit shown in FIG. 5 comprise a biological pharmaceutical composition receptacle component 640, a mixing receptacle component 605, a diluent component 645 filled with an appropriate amount of a diluent. Diluents in the diluent component 645 may be a medically relevant diluent such as an electrolyte solution, saline, buffered saline, dextrose, water, or combinations thereof. The amounts of diluent may range from 0 ml to approximately 1000 ml. The biological pharmaceutical preparation kit further may include a diluent transfer device 620, depicted in FIG. 5 as a syringe 620, but any transfer device that allows for the sterile transfer of the diluent may be used, for example, syringe, a flexible tube element, a pooling harness and a rigid tubing element.

The biological pharmaceutical preparation kit also may include a biological pharmaceutical composition conveying system 630. In FIG. 5, the conveying system 630 is depicted as a multi-lead multiple tubing system, such as, for example, a pooling harness set for pooling a plurality of the biological pharmaceutical compositions through tubing that is compatible with sterile connection technology, for example, a multi-lead multiple tubing system, a pooling harness set, a multi-lead rigid tubing element, a 4 to 1 spike adaptor and a 5 to 1 spike adaptor, among others. The kit may also may include a locking device 615 attached to the transfer device 620. As an example, the locking device 615 is shown as a threaded lock cannula. Further, the connector elements 610 and 615 are shown for connecting the transfer device 620 to both the mixing receptacle component 605 and the diluent component 645. The biological pharmaceutical preparation kit further comprises a human or an animal administration component (not shown) which may be, for example, an infusion pump, a syringe or a flexible tube that allows for administration via gravity flow.

An alternative embodiment of the biological pharmaceutical preparation kit is illustrated in FIG. 2. The components of the biological pharmaceutical preparation kit may comprise, for example, a biological pharmaceutical component 430, a mixing receptacle component 440, a diluent component 420 filled with an appropriate amount of a diluent. The biological pharmaceutical preparation kit also comprises a plurality of connector elements 415, 425, 435 that may be attached to the diluent component 420, the biological pharmaceutical component 430, and the mixing receptacle 440, respectively. As illustrated in FIG. 2, the connector elements 415, 425, 435 are depicted as a blood spike. It should be understood, any device that prevents contamination and can be repeatedly used without compromising the sterility of the contents of the components or receptacles may be substituted as the connector elements. The biological pharmaceutical preparation kit also comprises a locking device 410 and a transfer device 405.

In FIG. 2, for example, the locking device 410 is depicted as a threaded lock cannula. It should be understood that any locking device may be used that can be attached to the transfer device 405, and lock the transfer device 405 to the biological pharmaceutical component 430, the diluent component 420 and the mixing receptacle 440. Again, in FIG. 2, the transfer device 405, for example, is depicted as a syringe. The transfer device 405 may be any device, such as, for example, a flexible tubular conduit, capable of transferring the contents of the biological pharmaceutical component 430, the diluent component 420 and the mixing receptacle 440 outside of a BSC-type environment. The biological pharmaceutical preparation kit further comprises a human or an animal administration component (not shown).

Another aspect of the present technology comprises a method for preparing a biological pharmaceutical composition (e.g., MSC-based pharmaceutical compositions of the present technology) wherein the biological pharmaceutical composition is mixed with a diluent outside of a classified environment to form a biological pharmaceutical composition mixture.

Certain embodiments of the present technology may omit one or more of these steps and/or perform the steps in a different order than the order listed. For example, some steps may not be performed in certain embodiments of the present technology. As a further example, certain steps may be performed in a different temporal order, including simultaneously, than listed above. Additional steps as required to properly deliver various biological components that require such additional steps are also envisioned.

The foregoing descriptions of embodiments of the presently described technology have been presented for purposes of illustration and description. It is not intended to be exhaustive or to limit the presently described technology to the precise form disclosed, and modifications and variations are possible in light of the above teachings or may be acquired from practice of the present technology. The embodiments were chosen and described in order to explain the principals of the present technology and its practical application to enable one skilled in the art to utilize the present technology in various embodiments and with various modifications as are suited to the particular use contemplated.

Those skilled in the art will appreciate that the embodiments disclosed herein may be applied to the preparation of any biological pharmaceutical composition. Certain features of the embodiments of the claimed subject matter have been illustrated as described herein; however, many modifications, substitutions, changes and equivalents will now occur to those skilled in the art.

Additionally, while several functional blocks and relations between them have been described in detail, (e.g., FIGS. 2-6) it is contemplated by those of skill in the art that several of the operations may be performed without the use of the others, or additional functions or relationships between functions may be established and still be in accordance with the claimed subject matter. It is, therefore, to be understood that the appended claims are intended to cover all such modifications and changes as fall within the true spirit of the embodiments of the claimed subject matter.

Several embodiments are described above with reference to drawings. These drawings illustrate certain details of specific embodiments that implement the systems and methods of the present technology. However, describing the present technology with drawings should not be construed as imposing on the present technology any limitations associated with features shown in the drawings.

1. A method for preparation of a biological pharmaceutical composition mixture comprising the steps of:
transferring an appropriate amount of diluent from a diluent component to a mixing receptacle;
thawing a frozen biological pharmaceutical composition contained in a biological pharmaceutical component;
transferring diluent from the mixing receptacle to the biological pharmaceutical composition contained in the biological pharmaceutical component;
transferring the biological pharmaceutical composition and the diluent from the biological pharmaceutical component to the mixing receptacle;
mixing the biological pharmaceutical composition and the diluent to form a biological pharmaceutical composition mixture; and
administering an appropriate amount of the biological pharmaceutical composition mixture to a patient outside of a classified environment.

2. The method in claim 1, wherein the step of transferring the biological pharmaceutical from the biological pharmaceutical component to the mixing receptacle further comprises the steps of:
attaching a distal end of a conveying system to the biological pharmaceutical component; and
attaching a proximal end of the conveying system to the mixing receptacle.

3. The method of claim 2, wherein the conveying system is selected from a group consisting of a multi-lead multiple tubing system, a pooling harness, a 4 to 1 spike adaptor, a 5 to 1 spike adaptor, and a rigid tube system.

4. The method in claim 1, wherein the step of transferring the appropriate amount of diluent from the diluent component to the mixing receptacle further comprises the steps of:
attaching a first connector element to the diluent component;
attaching a second connector to the mixing receptacle;
connecting a transfer device to the first connector on the diluent component;
withdrawing an appropriate amount of the diluent into the transfer device from the diluent component; and
detaching the transfer device from the diluent component and attaching the transfer device to the second connector on the mixing receptacle.

5. The method of claim 4, wherein the transfer device is selected from a group consisting of a syringe, a flexible tube element, a pooling harness, and a rigid tube element.

6. The method of claim 1, wherein the appropriate amount of the biological pharmaceutical composition mixture is determined by a patient's weight.

7. The method of claim 6, wherein the patient is a human or an animal.

8. The method of claim 1, wherein the appropriate amount of the biological pharmaceutical composition mixture is based on a flat dose.

9. The method of claim 8, wherein the flat dose is comprised of at least one biological pharmaceutical composition contained in the biological pharmaceutical component for a human.

10. The method of claim 8, wherein the flat dose is comprised of at least one biological pharmaceutical composition contained in the biological pharmaceutical component for an animal.

11. The method in claim 4, wherein the first and second connector elements are selected from a group consisting of a blood spike and a coupling device.

12. The method of claim 1, wherein the biological pharmaceutical composition is comprised of multipotent or pluripotent cells.

13. The method of claim 1, wherein the biological pharmaceutical composition is comprised of differentiated cells.

14. The method of claim 12, wherein the multipotent or pluripotent cells are comprised of mesenchymal stem cells.

15. The method of claim 14, wherein the mesenchymal stem cells are comprised of human mesenchymal stem cells.

16. A method for preparing a biological pharmaceutical composition mixture comprising the steps of:
thawing a frozen biological pharmaceutical composition contained in a biological pharmaceutical component;
withdrawing an appropriate amount of a diluent from a diluent component into a transfer device;
transferring the diluent in the transfer device into the biological pharmaceutical component containing the biological pharmaceutical composition to form a biological pharmaceutical composition mixture;
withdrawing the biological pharmaceutical composition mixture from the biological pharmaceutical component into the transfer device;
transferring the biological pharmaceutical composition mixture in the transfer device into a mixing receptacle; and
administering an appropriate amount of the biological pharmaceutical composition mixture to a patient outside of a classified environment.

17. The method in claim 16, wherein the step of withdrawing the biological pharmaceutical composition mixture into the transfer device further comprises:
withdrawing a portion of the biological pharmaceutical composition mixture from the biological pharmaceutical component into the transfer device; and
transferring the portion of the biological pharmaceutical composition mixture in the transfer device back into the biological pharmaceutical component.

18. The method in claim 17, wherein the portion of the biological pharmaceutical composition mixture withdrawn from the biological pharmaceutical component ranges from 0%-100% by volume of the contents in the biological pharmaceutical component.

19. The method of claim 16, wherein the administration is by intravenous infusion.

20. The method of claim 16, wherein the administration is by intravenous injection.

21. The method of claim 16, wherein the transfer device is selected from a group consisting of a syringe, a flexible tubing element, a pooling harness, and a rigid tubing element.

22. A biological pharmaceutical preparation kit for preparing a biological pharmaceutical composition mixture outside of a classified environment comprising: a biological pharmaceutical component containing a biological pharmaceutical composition; a mixing receptacle; a diluent component filled with an appropriate diluent; a transfer device; a conveying system; and a human or an animal administration component.

23. The biological pharmaceutical preparation kit of claim 22, wherein the transfer device is selected from a group consisting of a syringe, a flexible tube element, a pooling harness, and a rigid tubing element.

24. The biological pharmaceutical preparation kit of claim 22, wherein the conveying system is selected from a group consisting of a multi-lead multiple tubing system, a pooling harness set, a multi-lead rigid tubing element, a 4 to 1 spike adaptor, and a 5 to 1 spike adaptor.

25. The biological pharmaceutical preparation kit of claim 22, wherein the biological pharmaceutical composition is comprised of multipotent or pluripotent cells.

26. The biological pharmaceutical preparation kit of claim 22, wherein the biological pharmaceutical composition is comprised of differentiated cells.

27. A method for preparing a biological pharmaceutical composition comprising mixing a biological pharmaceutical composition with a diluent outside of a classified environment to form a biological pharmaceutical composition mixture.
